SEQUENCE LISTING (39 sequences)

SEQ ID NO: 1 - protein, 32 aa, Artificial Sequence
  Description of Artificial Sequence Synthetic peptide
TTFDDVQAPNYTQHTSSMRGVYYPDEIFRSDT

SEQ ID NO: 2 - protein, 31 aa, Artificial Sequence
  Description of Artificial Sequence Synthetic peptide
FKDGIYFAATEKSNVVRGWVFGSTMNNKSQS

SEQ ID NO: 3 - protein, 31 aa, Artificial Sequence
  Description of Artificial Sequence Synthetic peptide
NSTNVVIRACNFELCDNPFFAVSKPMGTQTH

SEQ ID NO: 4 - protein, 28 aa, Artificial Sequence
  Description of Artificial Sequence Synthetic peptide
FEYISDAFSLDVSEKSGNFKHLREFVFK

SEQ ID NO: 5 - protein, 42 aa, Artificial Sequence
  Description of Artificial Sequence Synthetic peptide
DVSEKSGNFKHLREFVFKNKDGFLYVYKGYQPIDVVRDLPSG

SEQ ID NO: 6 - protein, 24 aa, Artificial Sequence
  Description of Artificial Sequence Synthetic peptide
KGYQPIDVVRDLPSGFNTLKPIFK

SEQ ID NO: 7 - protein, 36 aa, Artificial Sequence
  Description of Artificial Sequence Synthetic peptide
FSPAQDIWGTSAAAYFVGYLKPTTFMLKYDENGTIT

SEQ ID NO: 8 - protein, 23 aa, Artificial Sequence
  Description of Artificial Sequence Synthetic peptide
KYDENGTITDAVDCSQNPLAELK

SEQ ID NO: 9 - protein, 36 aa, Artificial Sequence; identical to SEQ ID NO: 7
  Description of Artificial Sequence Synthetic peptide

SEQ ID NO: 10 - protein, 31 aa, Artificial Sequence
  Description of Artificial Sequence Synthetic peptide
FVVKGDDVRQIAPGQTGVIADYNYKLPDDFM

SEQ ID NO: 11 - protein, 34 aa, Artificial Sequence
  Description of Artificial Sequence Synthetic peptide
NTRNIDATSTGNYNYKYRYLRHGKLRPFERDISN

SEQ ID NO: 12 - protein, 31 aa, Artificial Sequence
  Description of Artificial Sequence Synthetic peptide
FSPDGKPCTPPALNCYWPLNDYGFYTTTGIG

SEQ ID NO: 13 - protein, 34 aa, Artificial Sequence
  Description of Artificial Sequence Synthetic peptide
PKLSTDLIKNQCVNFNFNGLTGTGVLTPSSKRFQ

SEQ ID NO: 14 - protein, 31 aa, Artificial Sequence
  Description of Artificial Sequence Synthetic peptide
TPSSKRFQPFQQFGRDVSDFTDSVRDPKTSE

SEQ ID NO: 15 - protein, 39 aa, Artificial Sequence
  Description of Artificial Sequence Synthetic peptide
TNASSEVAVLYQDVNCTDVSTAIHADQLTPAWRIYSTGN

SEQ ID NO: 16 - protein, 35 aa, Artificial Sequence
  Description of Artificial Sequence Synthetic peptide
EHVDTSYECDIPIGAGICASYHTVSLLRSTSQKSI

SEQ ID NO: 17 - protein, 35 aa, Artificial Sequence; identical to SEQ ID NO: 16
  Description of Artificial Sequence Synthetic peptide

SEQ ID NO: 18 - protein, 40 aa, Artificial Sequence
  Description of Artificial Sequence Synthetic peptide
LKPTKRSFIEDLLFNKVTLADAGFMKQYGECLGDINARDL

SEQ ID NO: 19 - protein, 39 aa, Artificial Sequence
  Description of Artificial Sequence Synthetic peptide
NQKQIANQFNKAISQIQESLTTTSTALGKLQDVVNQNAQ

SEQ ID NO: 20 - protein, 39 aa, Artificial Sequence
  Description of Artificial Sequence Synthetic peptide
SKRVDFCGKGYHLMSFPQAAPHGVVFLHVTYVPSQERNF

SEQ ID NO: 21 - protein, 29 aa, Artificial Sequence
  Description of Artificial Sequence Synthetic peptide
EGKAYFPREGVFVFNGTSWFITQRNFFSP

SEQ ID NO: 22 - protein, 33 aa, Artificial Sequence
  Description of Artificial Sequence Synthetic peptide
DPLQPELDSFKEELDKYFKNHTSPDVDLGDISG

SEQ ID NO: 23 - protein, 30 aa, Artificial Sequence
  Description of Artificial Sequence Synthetic peptide
QKEIDRLNEVAKNLNESLIDLQELGKYEQY

SEQ ID NO: 24 - protein, 41 aa, Artificial Sequence
  Description of Artificial Sequence Synthetic peptide
LTVLPPLLTDDMIAAYTAALVSGTATAGWTFGAGAALQIPF

SEQ ID NO: 25 - protein, 40 aa, Artificial Sequence
  Description of Artificial Sequence Synthetic peptide
AMQMAYRFNGIGVTQNVLYENQKQIANQFNKAISQIQESL

SEQ ID NO: 26 - protein, 34 aa, Artificial Sequence
  Description of Artificial Sequence Synthetic peptide
ELDSFKEELDKYFKNHTSPDVDLGDISGINASVV

SEQ ID NO: 27 - protein, 37 aa, Artificial Sequence
  Description of Artificial Sequence Synthetic peptide
NIQKEIDRLNEVAKNLNESLIDLQELGKYEQYIKWPW

SEQ ID NO: 28 - protein, 27 aa, Artificial Sequence
  Description of Artificial Sequence Synthetic peptide
DSTDNNQNGGRNGARPKQRRPQGLPNN

SEQ ID NO: 29 - protein, 35 aa, Artificial Sequence
  Description of Artificial Sequence Synthetic peptide
GSRGGSQASSRSSSRSRGNSRNSTPGSSRGNSPAR

SEQ ID NO: 30 - protein, 34 aa, Artificial Sequence
  Description of Artificial Sequence Synthetic peptide
KVSGKGQQQQGQTVTKKSAAEASKKPRQKRTATK

SEQ ID NO: 31 - protein, 26 aa, Artificial Sequence
  Description of Artificial Sequence Synthetic peptide
GRRGPEQTQGNFGDQDLIRQGTDYKH

SEQ ID NO: 32 - protein, 34 aa, Artificial Sequence
  Description of Artificial Sequence Synthetic peptide
HIDAYKTFPPTEPKKDKKKKTDEAQPLPQRQKKQ

SEQ ID NO: 33 - protein, 35 aa, Artificial Sequence
  Description of Artificial Sequence Synthetic peptide
QKKQPTVTLLPAADMDDFSRQLQNSMSGASADSTQ

SEQ ID NO: 34 - protein, 81 aa, SARS coronavirus
AMQMAYRFNGIGVTQNVLYENQKQIANQFNKAISQIQESLLTVLPPLLTDDMIAAYTAALVSGTATAGWTFGAGAALQIPF

SEQ ID NO: 35 - protein, 71 aa, SARS coronavirus
ELDSFKEELDKYFKNHTSPDVDLGDISGINASVVNIQKEIDRLNEVAKNLNESLIDLQELGKYEQYIKWPW

SEQ ID NO: 36 - protein, 72 aa, SARS coronavirus
PELDSFKEELDKYFKNHTSPDVDLGDISGINASVVNIQKEIDRLNEVAKNLNESLIDLQELGKYEQYIKWPW

SEQ ID NO: 37 - protein, 71 aa, Human coronavirus
PNLPDFKEELDQWFKNQTLVAPDLSLDYINVTFLDLQDEMNRLQEAIKVLNQSYINLKDIGTYEYYVKWPW

SEQ ID NO: 38 - protein, 71 aa, Bovine coronavirus
PNLHDFKEELDQWFKNQTSVAPDLSLDYINVTFLDLQDEMNRLQEAIKVLNQSYINLKDIGTYEYYVKWPW

SEQ ID NO: 39 - protein, 73 aa, Mouse hepatitis virus
PNLPDFKEELDKWFKNQTSIAPDLSLDFEKLNVTFLDLTYEMNRIQDAIKKLNESYINLKEVGTYEMYVKWPW

(19) United States
(12) Patent Application Publication
(10) Pub. No.: US 2007/0092936 A1
(43) Pub. Date: Apr. 26, 2007

(54) Severe acute respiratory syndrome

(76) Inventors: Barton F. Haynes, Durham, NC (US); Hua-Xin Liao, Chapel Hill, NC (US)

Correspondence Address: NIXON & VANDERHYE, PC, 901 NORTH GLEBE ROAD, 11TH FLOOR, ARLINGTON, VA, 22203, US

(21) Appl. No.: 10/553,844

(22) PCT Filed: May 10, 2004
(86) PCT No.: PCT/US04/14512

§ 371 Date: Jun. 14, 2006

Related U.S. Application Data
(60) Provisional application No. 60/468,644, filed on May 8, 2003.

(51) Int. Cl. C12Q 1/68; C12P 21/06

(52) U.S. Cl. 435/69.1; 435/6

(57) ABSTRACT

The present invention relates, in general, to severe acute respiratory syndrome and, in particular, to a method of generating neutralizing antibodies to the virus. The invention further relates to methods of detecting the presence of the virus and to methods of treating infected individuals.

This application claims priority from U.S. Provisional Application No. 60/468,644, filed May 8, 2003, the entire content of which is incorporated herein by reference.

### TECHNICAL FIELD

The present invention relates, in general, to severe acute respiratory syndrome (SARS) and, in particular, to a method of generating neutralizing antibodies to the virus. The invention further relates to a method of detecting the presence of the virus and to a method of treating an infected individual.

### BACKGROUND

Since the severe acute respiratory syndrome (SARS) epidemic surfaced in Asia, more than 2600 cases have been identified in 19 countries, and more than 100 deaths have been reported. SARS has recently been identified as a new clinical entity (INFECTIOUS DISEASES: Deferring Competition, Global Net Closes In on SARS. Science 300(5617):224-5 (2003); Ksiazek et al, N. Engl. J. Med. Apr 10 (2003); Drosten et al, N. Engl. J. Med. Apr 10 [epub ahead of print] (2003); Poutanen et al, N. Engl. J. Med. Apr 10 [epub ahead of print] (2003)). It has been found that a novel coronavirus is associated with this outbreak, and the evidence indicates that this virus has an etiologic role in SARS since this virus was found in samples from multiple SARS patients in several independent laboratories. The complete genome of the SARS associated coronavirus (“the SARS virus”) was derived by sequencing of gene fragments generated using consensus coronavirus primers designed to amplify SARS genes by reverse transcription-polymerase chain reaction (RT-PCR).

The SARS virus is RNA virus with the genome size of approximately 29K nucleotides. The complete SARS virus genome sequence has been reported by Jones et al and is available in the NCBI DNA database (GI: 29826277). Phylogenetic analyses and sequence comparisons showed that the SARS virus is not closely related to any of the previously characterized coronaviruses (FIGS. 1-5).

### SUMMARY OF THE INVENTION

The present invention relates generally to SARS. More specifically, the invention relates to a method of producing neutralizing antibodies to the virus and to a method of treating individuals infected with the virus. The invention further relates to a method of detecting the presence of the virus in a sample. The invention additionally relates to compounds and compositions suitable for use in such methods.

Objects and advantages of the present invention will be clear from the description that follows.

### BRIEF DESCRIPTION OF THE DRAWINGS

FIG. 1. Amino acid sequence comparison of spike protein between SARS coronavirus with bovine coronavirus.

FIG. 2. Amino acid sequence comparison of spike proteins between SARS coronavirus with human coronavirus OC43.

FIG. 3. Phylogenetic analysis of coronavirus N protein. FIG. 4. Phylogenetic analysis of coronavirus S protein.

FIG. 5. Phylogenetic analysis of coronavirus M protein.

FIG. 6. Protein structure of SARS virus spike glycoprotein.

FIG. 7. Protein structure of SARS virus nucleocapsid (NP) protein.

FIG. 8. SARS spike protein peptides.

FIG. 9. SARS NP protein peptides.

FIG. 10. Coronavirus spike protein among isolates.

FIG. 11. Peptide design based on predicated SARS spike protein antigenic epitopes.

FIG. 12. HR and LZ domains in coronavirus spike proteins. (HR1 (SEQ ID NO:34), HR2 (SEQ ID NO:35))

FIG. 13. Immunization protocol of rabbits with SARS spike protein peptides.

FIG. 14. Schematic representation of SARS expression vectors.

FIG. 15. Western blot analysis of SARS spike protein, shown are purified SARS spike protein (lane 1), spike protein Ig fusion protein (lane 3) is and mock transfection supernatant control, produced in transformed 293 cells and purified using a lectin column—analysis was effected using Western blot and detection using immune sera of a mouse immunized with a DNA vaccine expressing SARS spike protein.

FIG. 16. Induction of antibody reacted with recombinant SARS spike protein by immunization with plasmid DNAs that express SARS-spike protein or spike protein-Ig. Serum samples were collected 10 days after immunizations and assayed by ELISA. Shown are the end-point ELISA titers against recombinant SARS spike proteins coated on a 96-well plate (200 ng/well).

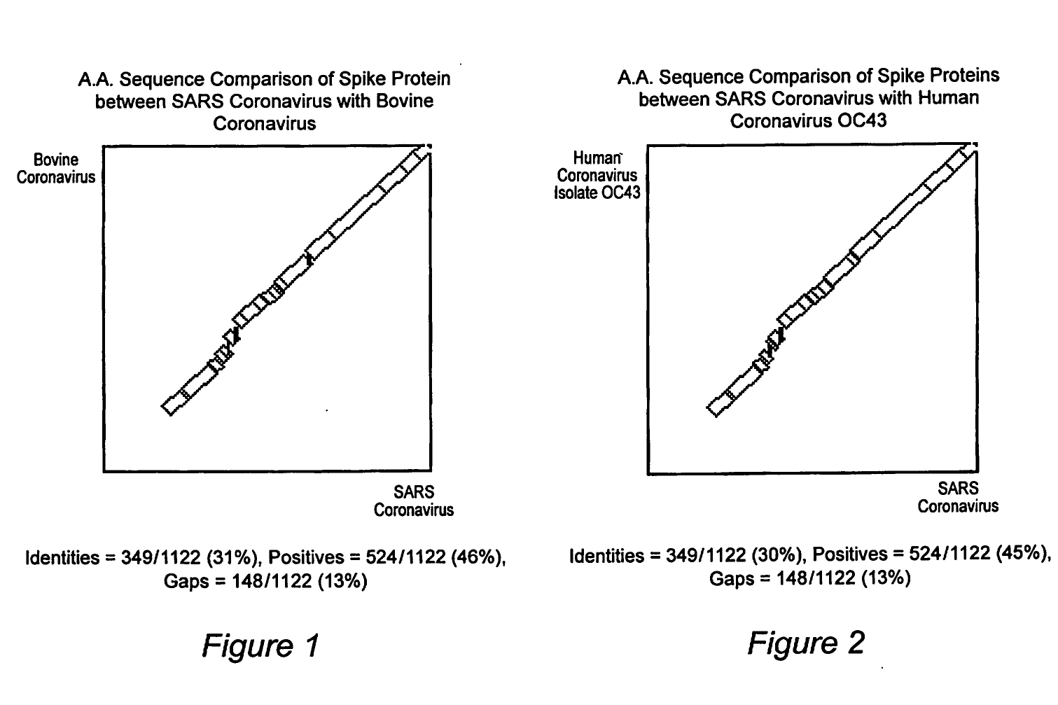

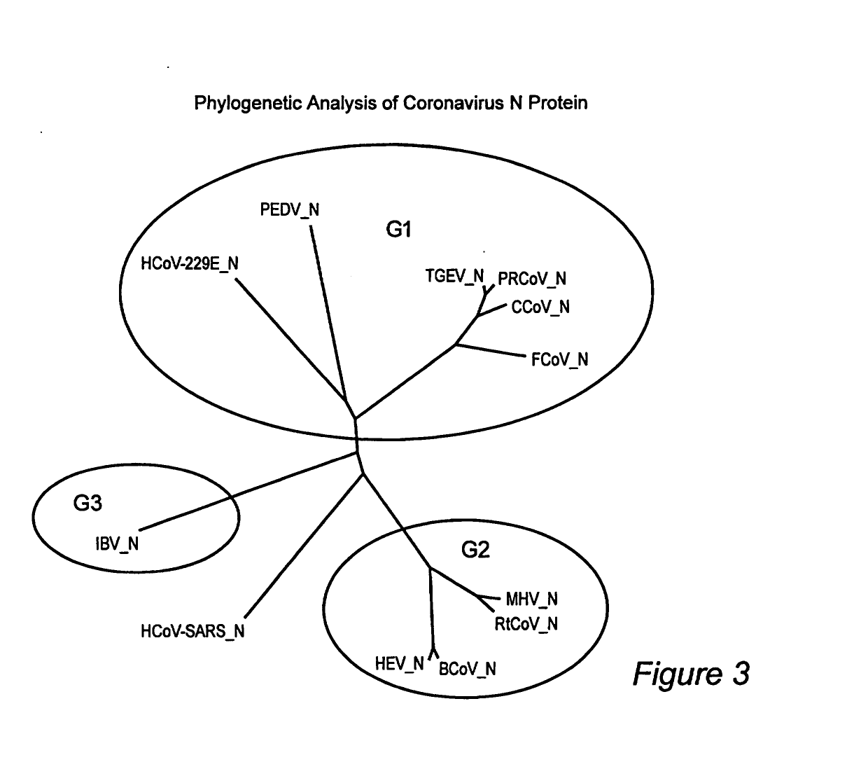

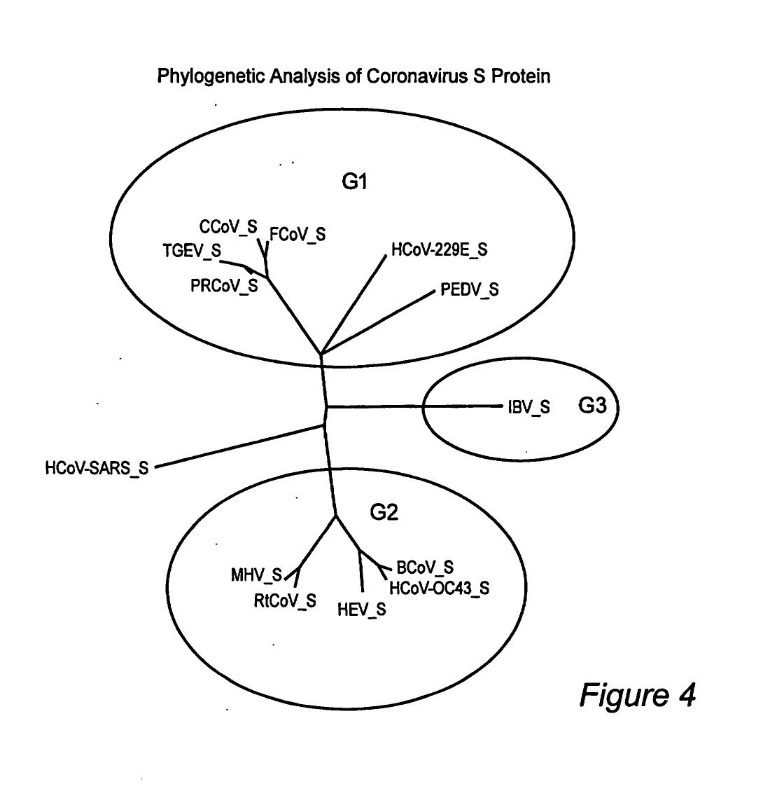

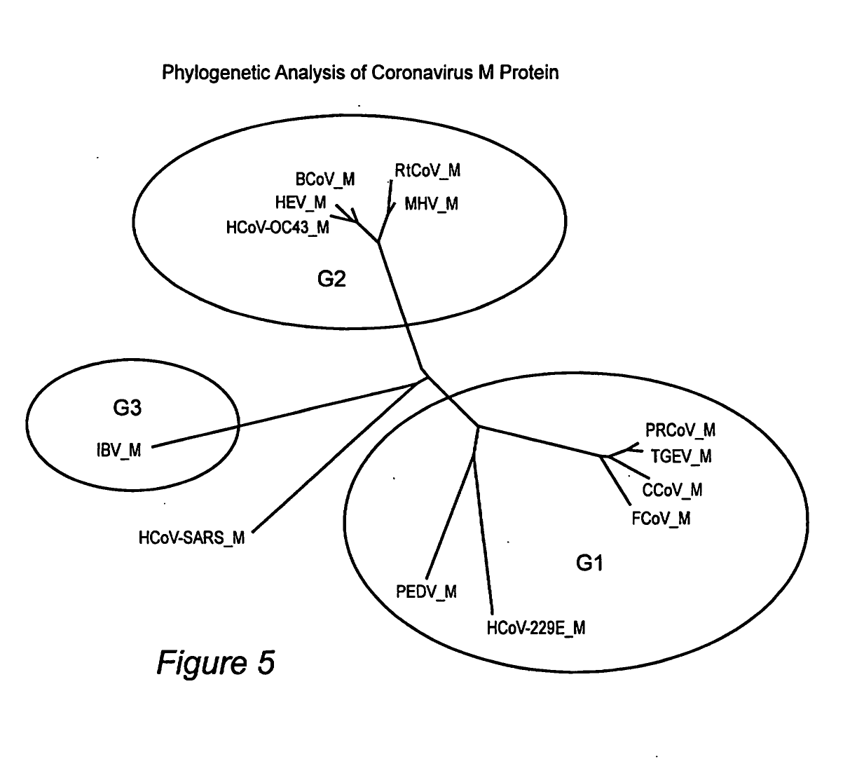

### DETAILED DESCRIPTION OF THE INVENTION

In one embodiment, the present invention relates to a method of producing neutralizing antibodies to the SARS virus. In a further embodiment, the invention relates to a method of treating an individual infected with the virus. In another embodiment, the invention relates to a method of detecting the presence of the SARS virus in a sample (e.g.. a biological sample). The invention also relates to compounds and compositions suitable for use in the such methods.

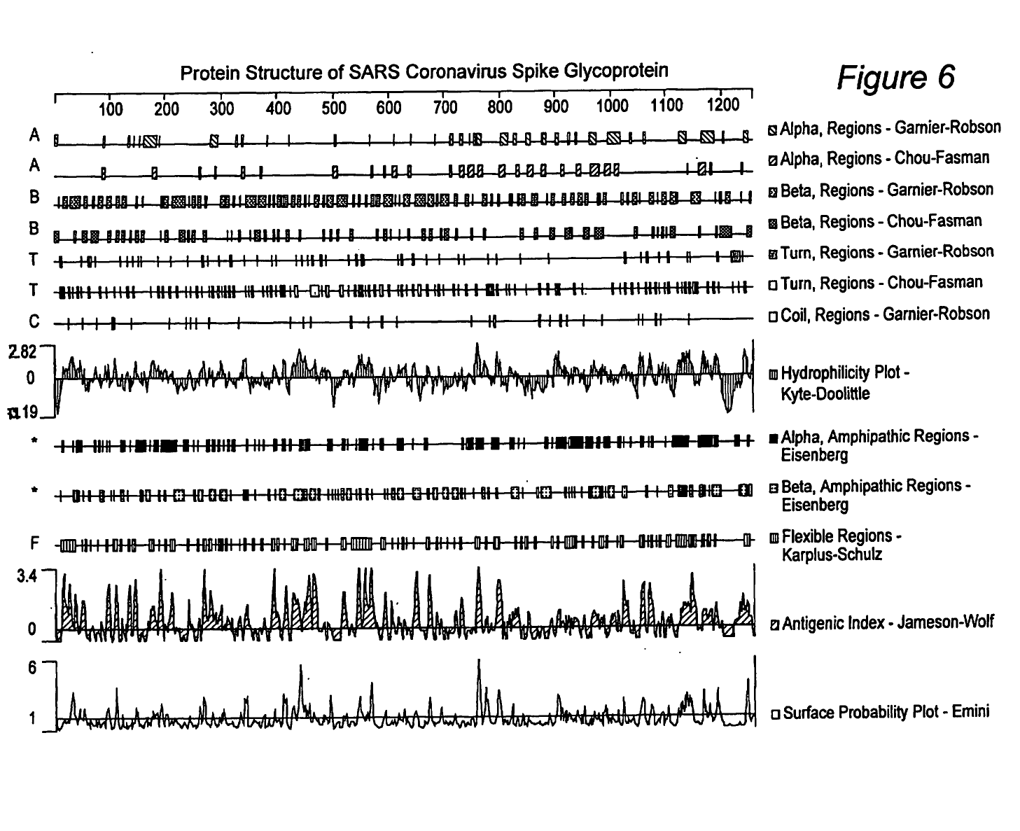

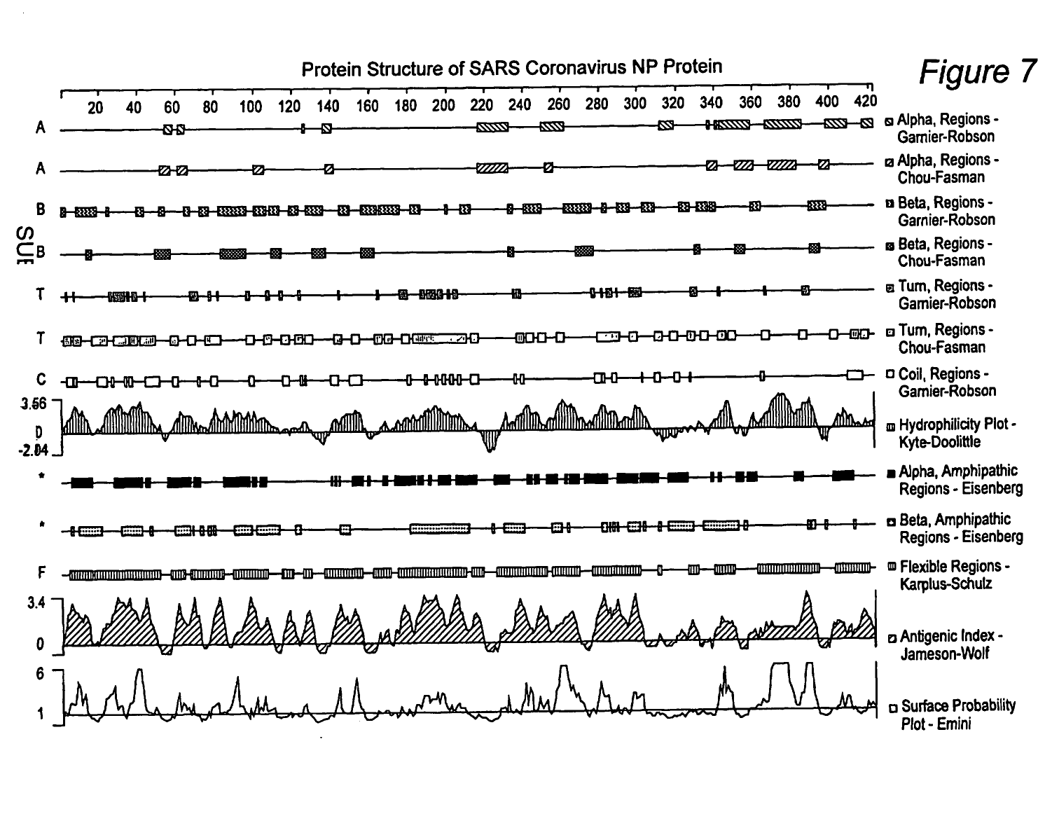

The structure of the SARS virus putative spike glycoprotein (1,255 amino acids) and that of the nucleocapsid protein (NP) (422 amino acids) have been analyzed using DNAStar computer program, version 3.16 (DNAStar Inc.) (see FIGS. 6 and 7, respectively; the notation on the right margin indicates the nature of the region such as antigenicity index, surface probability etc.).

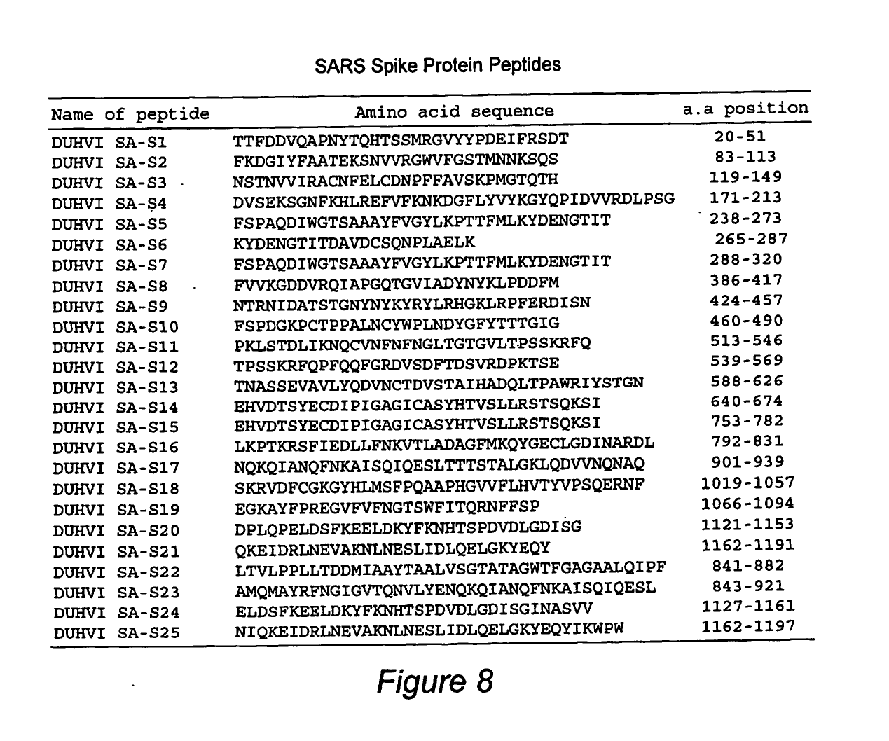

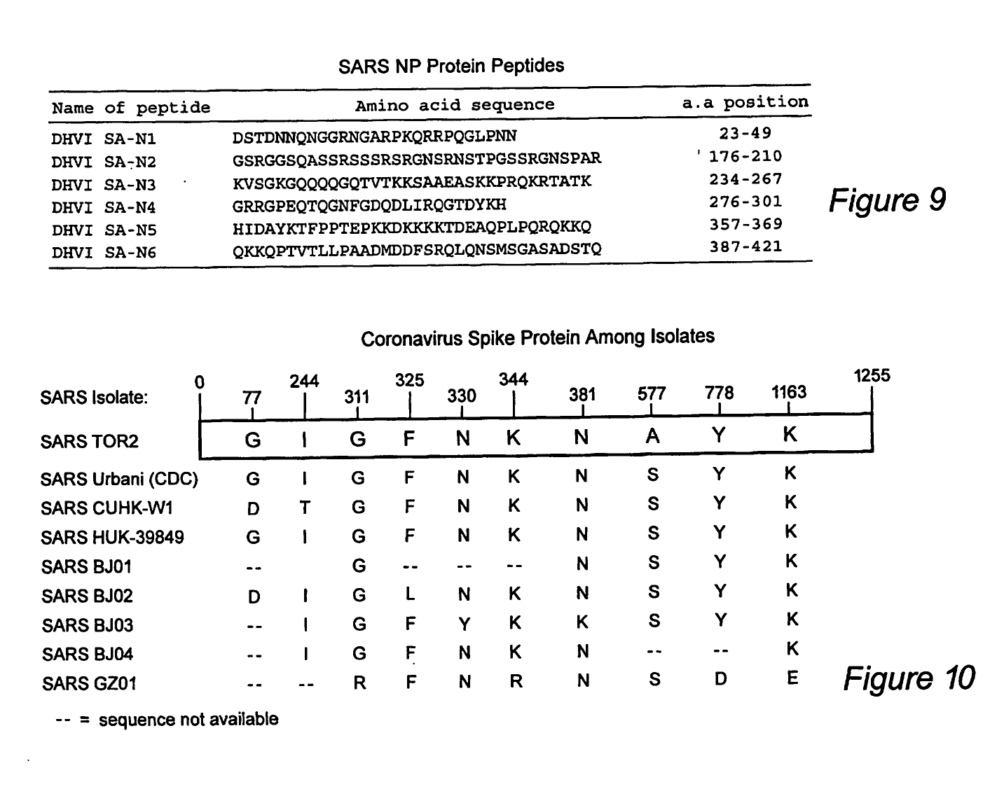

Based on the antigenic index of these two proteins, and data in the literature relating to other coronaviruses, the panel of peptides listed in Table 1 (SEQ ID NO:1 to SEQ ID NO:33, respectively) has been designed (see also FIG. 8 and 9). Positions of variability that have been identified in the SARS virus spike protein are shown in FIG. 10.

TABLE 1

Synthetic Peptides derived from SARS
coronavirus spike and N proteins.

| | |
|---|---|
| Name of | a.a |

| | | |
|---|---|---|
| peptide | Amino acid sequence | position |

| | | |
|---|---|---|
| DUHVI SA-S1 | TTFDDVQAPNYTQHTSSMRGVYYPDE | 20-51* |
| | IFRSDT | |
| DUHVI SA-S2 | FKDGIYFAATEKSNVVRGWVFGSTMN | 83-113 |
| | NKSQS | |
| DUHVI SA-S3 | NSTNVVIRACNFELCDNPFFAVSKPM | 119-149 |
| | GTQTH | |
| DUHVI SA-S4-A | FEYISDAFSLDVSEKSGNFKHLREFV | 161-188* |
| | FK | |
| DUHVI SA-S4 | DVSEKSGNFKHLREFVFKNKDGFLYV | 171-213 |
| | YKGYQPIDVVRDLPS | |
| DUHVI SA-S4-B | KGYQPIDVVRDLPSGFNTLKPIFK | 198-221* |
| DUHVI SA-S5 | FSPAQDIWGTSAAAYFVGYLKPTTFM | 238-273 |
| | LKYDENGTIT | |
| DUHVI SA-S6 | KYDENGTITDAVDCSQNPLAELK | 265-287* |
| DUHVI SA-S7 | FSPAQDIWGTSAAAYFVGYLKPTTFM | 288-320 |
| | LKYDENGTIT | |
| DUHVI SA-S8 | FVVKGDDVRQIAPGQTGVIADYNYKL | 386-417 |
| | PDDFM | |
| DUHVI SA-S9 | NTRNIDATSGNYNYKYRYLRHGKLRP | 424-457* |
| | FERDISN | |
| DUHVI SA-S10 | FSPDGKPCTPPALNCYWPLNDYGFYT | 460-490 |
| | TTGIG | |
| DUHVI SA-S11 | PKLSTDLIKNQCVNFNFNGLTGTGVL | 513-546 |
| | TPSSKRFQ | |
| DUHVI SA-S12 | TPSSKRFQPFQQFGRDVSDFTDSVRD | 539-569* |
| | PKTSE | |
| DUHVI SA-S13 | TNASSEVAVLYQDVNCTDVSTAIHAD | 588-626 |
| | QLTPAWRIYSTGN | |
| DUHVI SA-S14 | EHVDTSYECDIPIGAGICASYHTVSL | 640-674 |
| | LRSTSQKSI | |
| DUHVI SA-S15 | EHVDTSYECDIPIGAGICASYHTVSL | 753-782 |
| | LRSTSQKSI | |
| DUHVI SA-S16 | LKPTKRSFIEDLLFNKVTLADAGFMK | 792-831 |
| | QYGECLGDINARDL | |
| DUHVI SA-S17 | NQKQIANQFNKAISQIQESLTTTSTA | 901-939 |
| | LGKLQDVVNQNAQ | |
| DUHVI SA-S18 | SKRVDFCGKGYHLMSFPQAAPHGVVF | 1019-1057 |
| | LHVTYVPSQERNF | |
| DUHVI SA-S19 | EGKAYFPREGVFVFNGTSWFITQRNF | 1066-1094 |
| | FSP | |
| DUHVI SA-S20 | DPLQPELDSFKEELDKYFKNHTSPDV | 1121-1153* |
| | DLGDISG | |
| DUHVI SA-S21 | QKEIDRLNEVAKNLNESLIDLQELGK | 1162-1191 |
| | YEQY | |
| DUHVI SA-S22 | LTVLPPLLTDDMIAAYTAALVSGTAT | 841-882* |
| | AGWTFGAGAALQIPF | |
| DUHVI SA-S23 | AMQMAYRFNGIGVTQNVLYENQKQIA | 843-921* |
| | NQFNTAISQIQESL | |
| DUHVI SA-S24 | ELDSFKEELDKYFKNHTSPDVDLGDI | 1127-1161* |
| | SGINASVV | |
| DUHVI SA-S25 | NIQKEIDRLNEVAKNLNESLIDLQEL | 1162-1197* |
| | GKYEQYIKWPW | |
| DHVI SA-N1 | DSTDNNQNGGRNGARPKQRRPQGLPN | 23-49* |
| | N | |
| DHVI SA-N2 | GSRGGSQASSRSSSRSRGNSRNSTPG | 176-210* |
| | SSRGNSPAR | |
| DHVI SA-N3 | KVSGKGQQQQGQTVTKKSAAEASKKP | 234-267* |
| | RQKRTATK | |
| DHVI SA-N4 | GRRGPEQTQGNFGDQDLIRQGTDYKH | 276-301* |
| DHVI SA-N5 | HIDAYKTFPPTEPKKDKKKKTDEAQP | 357-369 |
| | LPQRQKKQ | |
| DHVI SA-N6 | QKKQPTVTLLPAADMDDFSRQLQNSM | 387-421 |
| | SGASADSTQ | |

The present invention includes the peptides set forth in Table 1 (and FIGS. 8 and 9), corresponding peptides from other SARS virus isolates and unique and/or antigenic portions of such peptides. Unique and/or antigenic portions are preferably at least 5 amino acids in length, more preferably, at least 6, 7, 8, 9 or 10 amino acids in length. The peptides can be synthesized, for example, using standard chemical syntheses techniques, as can polymers containing multiple copies of one or more of the above peptides or portions. The peptides (portions and polymers) can also be synthesized using well-known recombinant DNA techniques. Recombinant synthesis may be preferred when the peptides are covalently linked.

In addition to the above peptides (and portions and polymers), the invention also relates to nucleic acids encoding the same. The nucleic acids (e.g., DNA) can be present in a vector (e.g., a viral vector or a plasmid), advantageously linked to a promoter.

The invention includes compositions containing one or more of the above peptides (or portions or polymers), or nucleic acids encoding same, and a carrier, e.g., a pharmaceutically acceptable carrier. The peptide-containing compositions can further include an adjuvant (such as alum). The peptides of the invention (or portions or polymers) can be present in the composition conjugated to a carrier molecule, either directly or indirectly via a spacer molecule. Carrier molecules are, advantageously, non-toxic, pharmaceutically acceptable and of a size sufficient to produce an immune response in mammals. Examples of suitable carriers include tetanus toxoid and keyhole limpet hemocyanin.

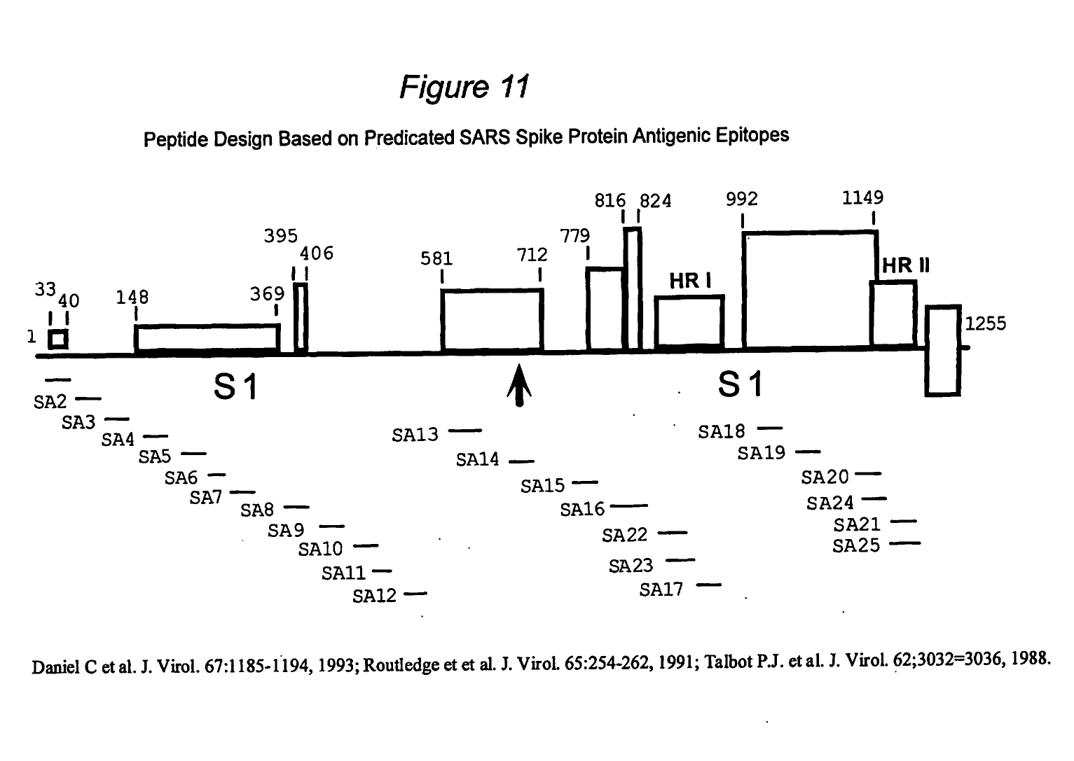

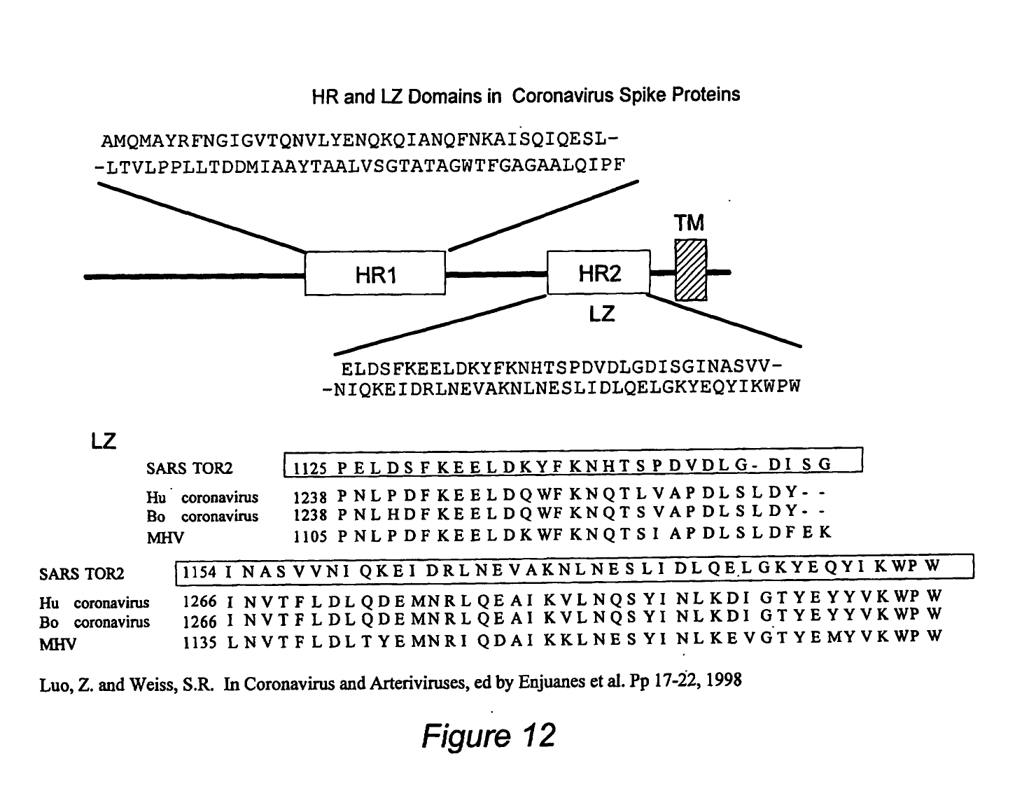

As indicated above, in one embodiment, the present invention relates to a method of producing neutralizing antibodies in a mammal (e.g., a human) to the SARS virus. The method comprises administering to a mammal in need thereof an amount of one or more of the above-described peptides, portions or polymers, sufficient to effect the production of neutralizing antibodies. (See also FIGS. 11 and 12—the regions specifically depicted in FIG. 11 corresponding to regions reportedly associated with the induction of neutralizing antibodies in the context of other coronaviruses; FIG. 12 provides the sequences of HR1 and HR2—these are sequences demonstrated to be capable of inhibiting fusion of animal coronaviruses (see Daniel et al, J. Virol. 67:1185-1194 (1993); Routledge et al, J. Virol. 65:254-262 (1991); Talbot et al. J. Virol 62:3032-3036 (1988) and Luo and Weiss In Coronavirus and Arteriviruses, ed. by Enjuanes, pp. 17-22 (1998)).) Optimum dosing regimens, which can vary with the peptide used, the patient and the effect sought, can be readily determined by one skilled in the art.

In an alternative aspect of this embodiment, production of neutralizing antibodies to the SARS virus can be effected by administering the above-described nucleic acids under conditions such that the nucleic acid is expressed, the encoded peptide produced and the neutralizing antibodies generated. That is, nucleic acids encoding the peptides (portions and polymers) of the invention can be used as components of, for example, a DNA vaccine wherein the peptide encoding sequence(s) is/are administered as naked DNA or, for example, a minigene encoding the peptides can be present in a viral vector. The encoding sequence(s) can be present, for example, in a replicating or non-replicating adenoviral vector, an adeno-associated virus vector, an attenuated mycobacterium tuberculosis vector, a Bacillus Calmette Guerin (BCG) vector, a vaccinia or Modified Vaccinia Ankara (MVA) vector, another pox virus vector, recombinant polio and other enteric virus vector, Salmonella species bacterial vector, Shigella species bacterial vector, Venezuelean Equine Encephalitis Virus (VEE) vector, a Semliki is Forest Virus vector, or a Tobacco Mosaic Virus vector. The encoding sequence(s), can also be expressed as a DNA plasmid with, for example, an active promoter such as a CMV promoter. Other live vectors can also be used to express the sequences of the invention. Expression of the peptides of the invention can be induced in a patient's own cells, by introduction into those cells of nucleic acids that encode the peptides, preferably using codons and promoters that optimize expression in human cells. Examples of methods of making and using DNA vaccines are disclosed in U.S. Pat. Nos. 5,580,859, 5,589,466, and 5,703,055.

In another embodiment, the present invention relates to a method of treating an individual (e.g., a human) infected with the SARS virus. As above, this method can be effected by administering the above-described peptides (portions and polymers) (the use of one or more of peptides SA-20 to SA-25 from Table 1, or portions thereof or polymers comprising same, being preferred) or nucleic acids in an amount and under conditions such that the treatment is effected. Peptides comprising HR-1 and/or HR-2, or portions thereof, are particulaly preferred. The significance of the HR-1 and HR-2 (LZ (leucine zipper)) regions is that these are homologous regions to the coil coil structures of HIV gp41, and HR-2 corresponds to the HR-2 or (T-20) drug that is working so well for HIV. Thus, the SARS virus HR-1 or HR-2 peptide (or portion thereof) can be expected to inhibit fusion of infected cells and prevent virus entry.

Optimum dosing regimens can be readily determined by one skilled in the art.

Suitable routes of administration of the peptides (portions and polymers) and nucleic acid of the invention include systemic (e.g. intramuscular or subcutaneous). Alternative routes can be used when an immune response is sought in a mucosal immune system (e.g., intranasal).

In another embodiment, the invention relates to methods of detecting the SARS virus in a sample (e.g., a biological sample from a patient, such as a blood, serum, sputum or fecal sample, or an environmental sample, such as a water or sewage sample). As appropriate, the method can be effected by detecting the presence of viral proteins or nucleic acids. For example, the above-described peptides (portions or polymers) can be used to generate antibodies (polyclonal or monoclonal) using standard techniques. The antibodies (or binding fragments thereof) can then be used, for example, in standard immunoassays, to detect the presence of SARS viral protein in the sample. The peptides (portions and polymers) can also be used, for example, in accordance with standard immunoassay techniques, to detect the presence of viral antibodies in, for example, the blood of a patient. Alternatively, the nucleic acids described above, or complements thereof, can be used according to standard techniques as probes or primers to detect the presence of viral encoding sequences in a sample. It will be appreciated that any of the peptides (portions or polymers), antibodies (or fragment) or nucleic acids can bear a detectable label (e.g., a fluorescent or radiolabel).

Certain aspects of the invention can be described in greater detail in the non-limiting Examples that follows.

### EXAMPLE 1

### Development of Polyclonal Immune Sera by Immunization in Rabbits with Synthetic Peptides Derived from SARS Virus

Peptides listed in Table 1 are synthesized as crude peptides, purified and analyzed. Rabbits (2 for each peptides) are immunized with this panel of SARS virus peptides at a dose of 250 μg per injection per animal for a total of 5 immunizations with RIBI adjuvant. Serum samples are collected 10 days after each immunization, and assayed against the immunizing peptides. Further characterization of immune sera including the reactivity of immune sera with native SARS virus proteins is effected.

### EXAMPLE 2

### Development of Monoclonal Antibodies Against the SARS Virus Spike Glycoprotein and NP Using Synthetic Peptides Derived the SARS Virus as Immunogen

Based on the initial immunogenicity results of the panel of SARS virus peptides, 1-2 peptides are selected from both SARS spike glycoprotein and NP as immunogens to immunize Balb/c mice for development of monoclonal antibodies. Immune sera and initial screening of hybridoma cell culture are carried out using the immunizing peptides. Further characterization and screening of monoclonal antibodies are effected using SARS native spike glycoprotein and NP expressed in a eukaryotic cell expression system. The neutralizing activities of the monoclonal antibodies are assessed.

### EXAMPLE 3

### Development of Polyclonal Immune Sera by Immunization of Rabbits with Synthetic Peptides Derived from SARS Coronavirus

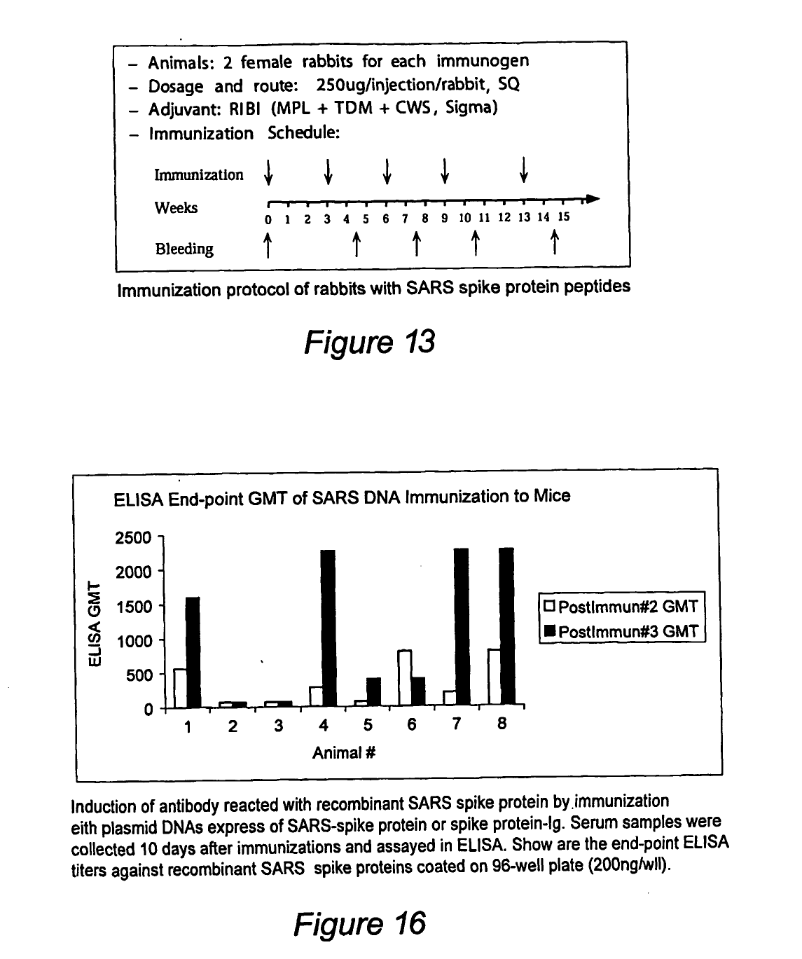

The protein structure of the putative spike glycoprotein (1,255 amino acids) has been analyzed using DNAStar computer program. Based on the antigenic index of these two proteins, a panel of 33 peptides derived from SARS coronavirus spike protein and NP proteins (as listed in Table 1) has been designed. Of these peptides, nine (S1, S4A, S4B, S9, S12, S20, S23. S24 and S25) have been used to immunize rabbits using a immunization protocol as shown in FIG. 13. Other peptides will be used in the future experiments.

### EXAMPLE 4

### Expression of SARS Coronavirus Spike Glycoprotein and Development of Monoclonal Antibodies (Mabs) Against SARS Virus

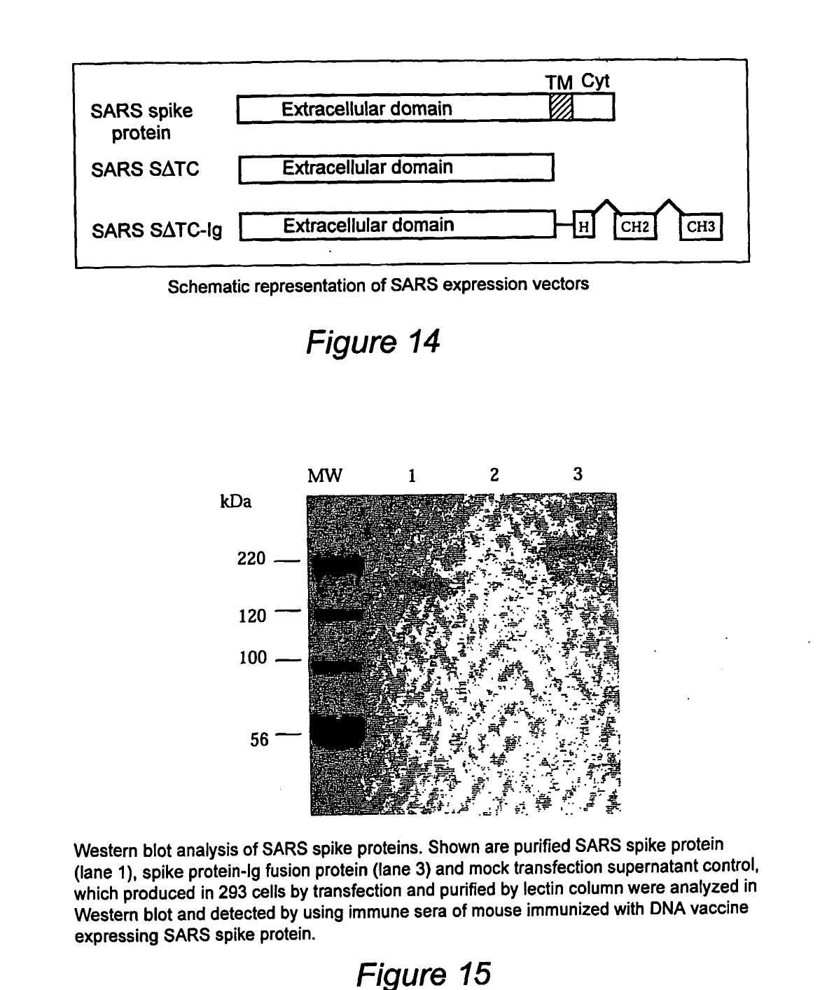

To develop Mabs and vaccine immunogens against SARS virus, a SARS coronavirus spike protein gene has been developed with codon- and RNA structure optimized for optimal expression. To produce secreted soluble SARS spike protein, an expression vector (SARS SΔTC) was generated in which the transmembrane (TM) and cytoplasmic domain (Cyt) of SARS spike protein was deleted. To enhance the immunogenicity and stability as well as to provide for ease of purification of SARS spike protein, the extracellular domain of SARS spike protein was linked with either mouse or human IgG constant region genomic sequence (FIG. 14). These 2 vectors were used for production of spike protein in vitro by transfection and also used as vaccine immunogens for development of monoclonal antibody as well as vaccine immunogens for induction of neutralizing antibodies against SARS virus.

As shown in FIG. 15, SARS spike proteins have been expressed in 293 cells by transfection with SARS SΔTC and SARSΔTC-Ig vectors and purified using a lectin column. Purified proteins were analyzed by SDS-PAGE and Western blot (FIG. 15). The extracellular domain SARS spike protein has a molecular weight of approximately 150 Kda, and SARS spike protein-Ig fusion protein has a molecular weight of approximately 170 Kda as detected by immune serum from a mouse immunized with the DNA vaccine that expresses SARS spike protein extracellular domain (FIG. 14). The purified SARS spike protein has been used for evaluation of immunogenicity of SARS spike protein expression DNA vaccine (see below). To generate Mabs, mice (4 mice for each group) have been immunized with the SARS SΔTC vector that expresses SARS spike protein. Mice developed antibody responses as detected using Western blot (FIG. 15) and ELISA (FIG. 16). Both SARS SΔTC and SARSΔTC-Ig vectors were also used as DNA vaccine immunogens for evaluation of the immunogenicity for induction of neutralizing antibody against SARS.

All documents cited above are hereby incorporated in their entirety by reference.

1. A method of producing, in a mammal, antibodies that neutralize severe acute respiratory syndrome (SARS) coronavirus, said method comprising administering to said mammal at least one peptide comprising amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, or 1162-1197 of SARS coronavirus spike protein, or amino acids 23-49, 176-210, 234-267, 276-301, 357-369 or 387-421 of SARS coronavirus N protein, or antigenic fragment thereof, in an amount such that said production is effected.

2. The method according to claim 1 wherein said at least one peptide comprises an amino acid sequence selected from the group consisting of those set forth in forth in SEQ ID NO:1 to SEQ ID NO:27, and antigenic fragments thereof.

3. The method according to claim 1 wherein said at least one peptide comprises an amino acid sequence selected from the group consisting of those set forth in SEQ ID No:28 to SEQ ID No:33, and antigenic fragments thereof.

4-5. (canceled)

6. The method according to claim 1 wherein said at least one peptide comprises at least two copies of amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, or 1162-1197 of SARS coronavirus spike protein, or amino acids 23-49, 176-210, 234-267, 276-301, 357-369 or 387-421 of SARS coronavirus N protein, or antigenic fragment thereof.

7. The method according to claim 1 wherein said at least one peptide comprises at least two different amino acid sequences selected from the group consisting of amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, and 1162-1197 of SARS coronavirus spike protein, and amino acids 23-49, 176-210, 234-267, 276-301, 357-369 and 387-421 of SARS coronavirus N protein, and antigenic fragments thereof.

8. A method of producing, in a mammal, antibodies that neutralize SARS coronavirus, said method comprising administering to said mammal at least one peptide comprising amino acids 33-40, 148-369, 395-406, 581-712, 779-816, 816-824 or 992-1149 of SARS coronavirus spike protein, or antigenic fragment thereof, in an amount such that said production is effected, or at least one peptide comprising HR-1 or HR-2 of SARS coronavirus spike protein, or antigenic fragment thereof, in an amount such that said production is effected.

9. (canceled)

10. The method according to claim 1 or 8 wherein said administration is effected by administering to said mammal at least one nucleic acid sequence encoding said at least one peptide under conditions such that said nucleic acid is expressed and said peptide is thereby produced.

11-14. (canceled)

15. A method of inhibiting fusion of SARS coronavirus to cells of a mammal, said method comprising administering to said mammal at least one peptide comprising HR-1 or HR-2 of SARS coronavirus spike protein, or portion thereof that inhibits said fusion, in an amount sufficient to effect said inhibition.

16. The method according to claim 15 wherein said at least one peptide comprises the amino acid sequence set forth in SEQ ID NO:34 or SEQ ID NO:35, or portion thereof that inhibits said fusion.

17. A composition comprising at least one peptide comprising amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, or 1162-1197 of SARS coronavirus spike protein or amino acids 23-49, 176-210, 234-267, 276-301, 357-369 or 387-421 of SARS coronavirus N protein, or antigenic fragment thereof, and a carrier.

18. The composition according to claim 17 wherein said at least one peptide comprises at least two copies of amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, or 1162-1197 of SARS coronavirus spike protein, or amino acids 23-49, 176-210, 234-267, 276-301, 357-369 or 387-421 of SARS coronavirus N protein, or antigenic fragment thereof.

19. The composition according to claim 17 wherein said at least one peptide comprises at least two different amino acid sequences selected from the group consisting of amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, and 1162-1197 of SARS coronavirus spike protein, and amino acids 23-49, 176-210, 234-267, 276-301, 357-369 and 387-421 of SARS coronavirus N protein, and antigenic fragments thereof.

20-21. (canceled)

22. A composition comprising at least one peptide comprising amino acids 33-40, 148-369, 395-406, 581-712, 779-816, 816-824 or 992-1149 of SARS coronavirus spike protein, or antigenic fragment thereof, or at least one peptide comprising HR-1 or HR-2 of SARS coronavirus spike protein, or antigenic fragment thereof or portion thereof that inhibits fusion, and a carrier.

23. (canceled)

24. An isolated nucleic acid sequence encoding amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, or 1162-1197 of SARS coronavirus spike protein, or amino acids 23-49, 176-210, 234-267, 276-301, 357-369 or 387-421 of SARS coronavirus N protein, or antigenic fragments thereof, or complement thereof.

25. An isolated nucleic acid sequence encoding amino acids 33-40, 148-369, 395-406, 581-712, 779-816, 816-824 or 992-1149 of SARS coronavirus spike protein, or antigenic fragment thereof, or complement thereof, or encoding HR-1 or HR-2 of SARS coronavirus spike protein, or antigenic fragment thereof or portion thereof that inhibits fusion, or complement thereof.

26. (canceled)

27. An antibody, or binding fragment thereof, specific for amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, or 1162-1197 of SARS coronavirus spike protein, or amino acids 23-49, 176-210, 234-267, 276-301, 357-369 or 387-421 of SARS coronavirus N protein, or antigenic fragment thereof.

28. An antibody, or binding fragment thereof, specific for amino acids 33-40, 148-369, 395-406, 581-712, 779-816, 816-824 or 992-1149 of SARS coronavirus spike protein, or antigenic fragment thereof, or specific for HR-1 or HR-2 of SARS coronavirus spike protein, or antigenic fragment thereof.

29. (canceled)

30. A method of detecting SARS coronavirus protein in a sample comprising contacting said sample with said antibody, or binding fragment thereof, according to claim 27 or 28, under conditions such that said antibody can bind to said protein and detecting the presence of a complex comprising said antibody and said protein.

31. A method of detecting antibodies to SARS coronavirus protein in a sample comprising contacting said sample with at least one peptide comprising an amino acid sequence selected from the group consisting of amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, and 1162-1197 of SARS coronavirus spike protein, and amino acids 23-49, 176-210, 234-267, 276-301, 357-369 and 387-421 of SARS coronavirus N protein, and antigenic fragments thereof, under conditions such that said peptide can bind to said antibodies and detecting the presence of a complex comprising said antibodies and said peptide.

32. A method of detecting antibodies to SARS coronavirus protein in a sample comprising contacting said sample with: i) at least one peptide comprising an amino acid sequence selected from the group consisting of amino acids 33-40, 148-369, 395-406, 581-712, 779-816, 816-824 or 992-1149 of SARS coronavirus spike protein, and antigenic fragments thereof, or ii) at least one peptide comprising HR-1 or HR-2 of SARS coronavirus spike protein, or antigenic fragment thereof, under conditions such that said peptide can bind to said antibodies and detecting the presence of a complex comprising said antibodies and said peptide.

33. (canceled)

34. A method of detecting the presence of a SARS coronavirus encoding sequence in a sample comprising contacting said sample with the nucleic acid sequence according to claim 24 or 25, or complement thereof, and detecting the formation of a complex between said nucleic acid sequence, or complement thereof, and said encoding sequence.

35. An isolated peptide comprising an amino acid sequence selected from the group consisting of amino acids 20-51, 83-113, 119-149, 161-188, 171-213, 198-221, 238-273, 265-287, 288-320, 386-417, 424-457, 460-490, 513-546, 539-569, 588-626, 640-674, 753-782, 792-831, 901-939, 1019-1057, 1066-1094, 1121-1153, 1162-1191, 841-882, 843-921, 1127-1161, and 1162-1197 of SARS coronavirus spike protein and amino acids 23-49, 176-210, 234-267, 276-301, 357-369 and 387-421 of SARS coronavirus N protein, and antigenic fragments thereof.

36. (canceled)

37. An isolated peptide comprising an amino acid sequence selected from the group consisting of amino acids 33-40, 148-369, 395-406, 581-712, 779-816, 816-824 and 992-1149 of SARS coronavirus spike protein, and antigenic fragments thereof, or comprising HR-1 or HR-2 of SARS coronavirus spike protein, or antigenic fragment thereof or portion thereof that inhibits fusion.

38. (canceled)
